(19) United States
(12) Patent Application Publication
(10) Pub. No.: US 2014/0091926 A1
(43) Pub. Date: Apr. 3, 2014

(54) SYSTEM AND METHOD FOR REDUCING MEDICAL ERROR

(71) Applicants: Jonathan Peter Gips, Hingham, MA (US); Aaron Douglas Valade, Hong Kong (CN); Philip Angus Liang, Hong Kong (CN); Ryan Patrick Aylward, Cambridge, MA (US)

(72) Inventors: Jonathan Peter Gips, Hingham, MA (US); Aaron Douglas Valade, Hong Kong (CN); Philip Angus Liang, Hong Kong (CN); Ryan Patrick Aylward, Cambridge, MA (US)

(21) Appl. No.: 14/115,357

(22) PCT Filed: May 3, 2012
(86) PCT No.: PCT/IB12/52207

§ 371 Date: Dec. 16, 2013

(30) Foreign Application Priority Data
May 4, 2011 (HK) 11104428.1

(51) Int. Cl. A61B 19/00; G08B 1/08

(52) U.S. Cl. CPC A61B 19/44; G08B 1/08; A61B 19/36
USPC 340/539.12

(57) ABSTRACT

A system and method for reducing medical error is disclosed. In one embodiment, the system comprises a worker device adapted to be worn on a worker, a compliance device, an action device and a base station. The compliance device defines a work zone based on a signal strength received by the worker device from a monitoring signal transmitted from the compliance device. The action device is adapted to be installed to a pump bottle, having a pressure-sensitive mechanism for actuating the action device upon the worker pressing the pump bottle, and an omnidirectional antenna adapted to transmit an action signal to be received by the worker device upon actuation of the action device. The base station is adapted to receive data transmitted from said worker device.

### FIELD OF INVENTION

This invention relates to a system and method for reducing medical error, and in particular a system and method for determining whether a medical worker has complied with a pre-determined protocol.

### BACKGROUND OF INVENTION

Medical error greatly increases the operation costs of hospitals. In particular, human medical errors due to a medical worker not following a protocol make up a significant portion of the increase. Therefore, a system and method to reduce such human medical errors is desired.

### SUMMARY OF INVENTION

In the light of the foregoing background, it is an object of the present invention to provide an alternate system and method for reducing medical error.

Accordingly, the present invention, in one aspect, is a system for reducing medical error, comprising a worker device adapted to be worn on a worker, a compliance device, an action device and a base station. The worker device has a directional antenna working as a transmitter and a receiver, a battery, and a memory for storing data. The compliance device has a directional antenna working as a transmitter, the directional antenna defines a work zone relative to the compliance device based on a signal strength received by the worker device from a monitoring signal transmitted from the compliance device. The action device is adapted to be installed to a pump bottle, having a pressure-sensitive mechanism for actuating the action device upon the worker pressing the pump bottle, and an omnidirectional antenna adapted to transmit an action signal to be received by the worker device upon actuation of the action device. The base station is adapted to receive the data transmitted from the worker device.

In an exemplary embodiment of the present invention, the battery of the worker device is rechargeable through a battery charger.

In a further embodiment of the present invention, the battery charger comprises a slot for insertion of the battery of the worker device, wherein at least one stopper is provided within the slot to align the battery to the battery charger.

In an embodiment of the present invention, the battery of the worker device is detachable from the worker device.

In an exemplary embodiment of the present invention, the antenna of the worker device is a patch antenna oriented vertically to achieve horizontal directionality.

In another exemplary embodiment of the present invention, the antenna of the compliance device is a patch antenna oriented vertically to achieve horizontal directionality.

In an exemplary embodiment of the present invention, the antenna of the action device comprises a plurality of quarter-wave whip antennas oriented vertically in a circularly symmetric configuration to achieve horizontal omnidirectionality.

In a further embodiment of the present invention, the plurality of whip antennas is configured in a way such that only one of the plurality of whip antennas is active at a given time.

In yet another exemplary embodiment of the present invention, a ground plane is located at a bottom end of the antenna of the action device.

In a further embodiment, a distance of at least 35 mm is provided between the ground plane and a top surface of the pressure-sensitive mechanism.

In another embodiment, the pressure-sensitive mechanism comprises a movable platform adapted to move to a depressed position upon exertion of pressure, the movable platform at the depressed position activates a switch for transmitting the action signal.

According to another aspect of the present invention, a method of reducing medical errors is disclosed. In a system as described in paragraph [0004] above, the method provides the worker device to a worker and detects the worker entering the work zone based on the signal strength of the monitoring signal. The method also detects activation of the pump bottle by the worker based on the action signal and detects the worker exiting the work zone based on the signal strength of the monitoring signal. A compliance rate is determined based on details of the detecting steps.

In an exemplary embodiment of the present invention, the step of detecting activation of the pump bottle comprises the steps of sensing a pressure exerted on the pump bottle by moving a movable platform of the pressure-sensitive mechanism to a depressed position, and activating a switch for transmitting the action signal to the worker device when the movable platform is at the depressed position.

In another embodiment, the pump bottle comprises a plurality of whip antennas, and the step of detecting activation of the pump bottle further comprises the step of configuring the plurality of antennas such that only one of the plurality of antennas transmits the action signal at a given time.

In yet another embodiment, the step of detecting the worker entering the work zone comprises the step of detecting the worker entering the work zone when the signal strength of the monitoring signal exceeds a first threshold, and the step of detecting the worker exiting the work zone comprises the step of detecting the worker exiting the work zone when the signal strength of the monitoring signal drops below a second threshold, wherein the first threshold is higher than the second threshold.

There are many advantages to the present invention. A main advantage of the present invention is that the monitoring of whether workers have followed a specific protocol is automated through this system. The system detects the time the worker performed every single step in the protocol, and determines whether the worker followed the protocol e.g. in the correct sequence or within a specified time frame. Labor cost can then be greatly reduced while the efficiency and reliability is increased. For example, by providing appropriate devices to define a work zone and also to detect a depression of a pump bottle, the system can determine whether a worker has washed his/her hands before touching a patient in a hospital.

Another advantage of the present invention is that the antennas of each component of the system are specifically designed with customized dimensions and directionality to optimize the detection while reducing false activation to a minimum. For example, the worker device and the compliance device have directional antennas to ensure detection on the worker walking forward into a work zone. The size of the work zone is also determined by the dimensions and the directionality of the antennas.

### BRIEF DESCRIPTION OF FIGURES

FIG. 1 is a block diagram of a system for reducing medical error, according to an embodiment of the present invention.

FIG. 2a is a front perspective view of a worker device according to an embodiment of the present invention.

FIG. 2b is a side view of the worker device as shown in FIG. 2a.

FIG. 3a is a front perspective view of a compliance device according to an embodiment of the present invention.

FIG. 3b is a back perspective view of the compliance device as shown in FIG. 3a.

FIG. 4 is a front perspective view of an action device according to an embodiment of the present invention.

FIG. 5 is a front perspective view of the pressure-sensitive mechanism of the action device in FIG. 4, according to an embodiment of the present invention.

FIG. 6 is a back perspective view of a battery charger according to an embodiment of the present invention with batteries of worker devices inserted therein.

### DETAILED DESCRIPTION OF THE PREFERRED EMBODIMENTS

As used herein and in the claims, “comprising” means including the following elements but not excluding others.

As used herein and in the claims, “couple” or “connect” refers to electrical coupling or connection either directly or indirectly via one or more electrical means unless otherwise stated.

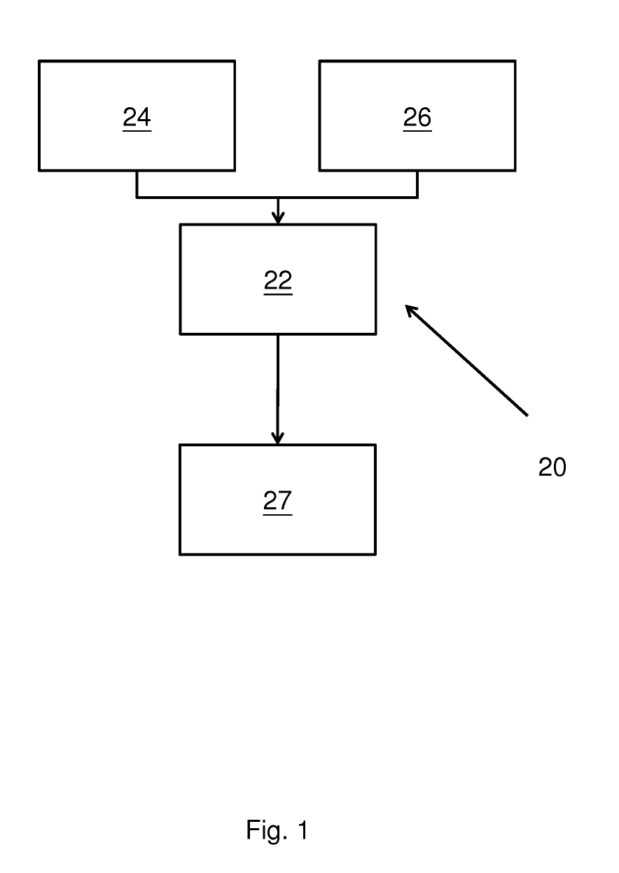

Referring now to FIG. 1, the first embodiment of the present invention is a system 20 for reducing medical error, comprising a worker device 22, a compliance device 24, an action device 26 and a base station 27. The compliance device 24 and the action device 26 transmit signals to the worker device 22, and the worker device 22 transmits a signal to the base station 27. In the following embodiments, the worker device 22 is also referred to as a badge, and the compliance device 24 is also called a beacon.

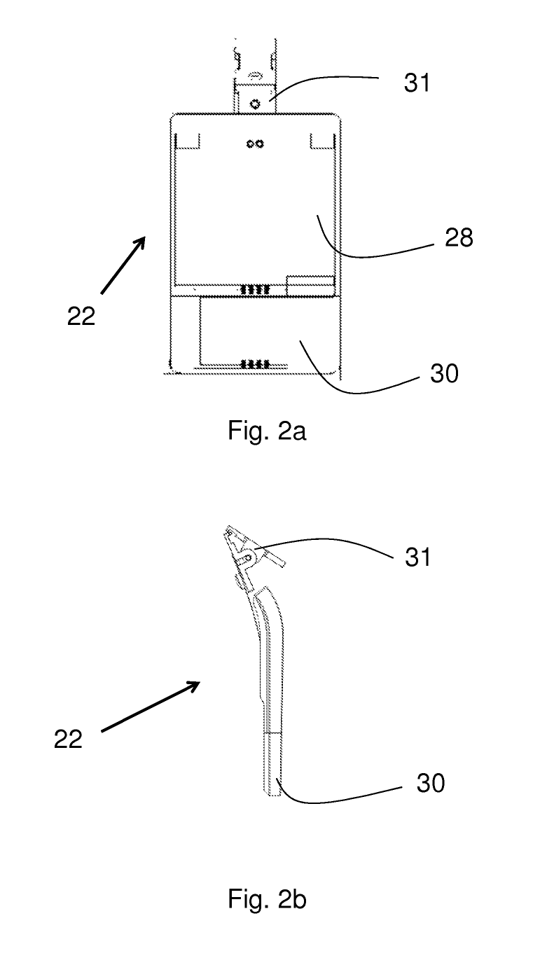

In an exemplary embodiment as shown in FIGS. 2a and 2b, the worker device 22 comprises a patch antenna 28 towards the front end of the device, a battery 30 and an attachment member 31. A patch antenna is a directional antenna which a maximum gain is achieved along an axis perpendicular to the plane of the antenna (i.e. 90 degrees), and is reduced when the angle approaches 0 or 180 degrees or behind the antenna (270 degrees). The directionality of the patch antenna 28 is related to the size of the antenna. Generally, when the antenna is smaller (in terms of wavelength), the directionality increases, meaning the gain will fall off quicker when the angle changes.

In a specific embodiment, the patch antenna 28 has a 3-dB beam width of 90 degrees. Having such a beam width eliminates variation in signal strength caused by the environment behind the patch antenna 28, including the worker's body.

In an exemplary embodiment, the battery 30 is a rechargeable battery such as a lithium polymer battery. In a specific embodiment, the rechargeable battery 30 is detachable from the other parts of the worker device 22 such as the patch antenna 28 and other electronic circuitry. In a further embodiment, the rechargeable battery 30 is configured to snap into contact with the worker device 22 at one end of the worker device 22. As such, the worker device 22 does not need to be opened when replacing the battery 30.

In an exemplary embodiment, the attachment member 31 extends from the back surface of the worker device 22. In a specific embodiment, the attachment member 31 comprises a strap attached to the worker device 22 and a clip at an end of the strap for clipping to the worker. In an exemplary embodiment, from the front perspective view, the worker device 22 is designed to be slightly bent inwards at a top end of the device. This makes the worker device 22 more ergonomic to better fit the body contour of the worker when attached to the worker.

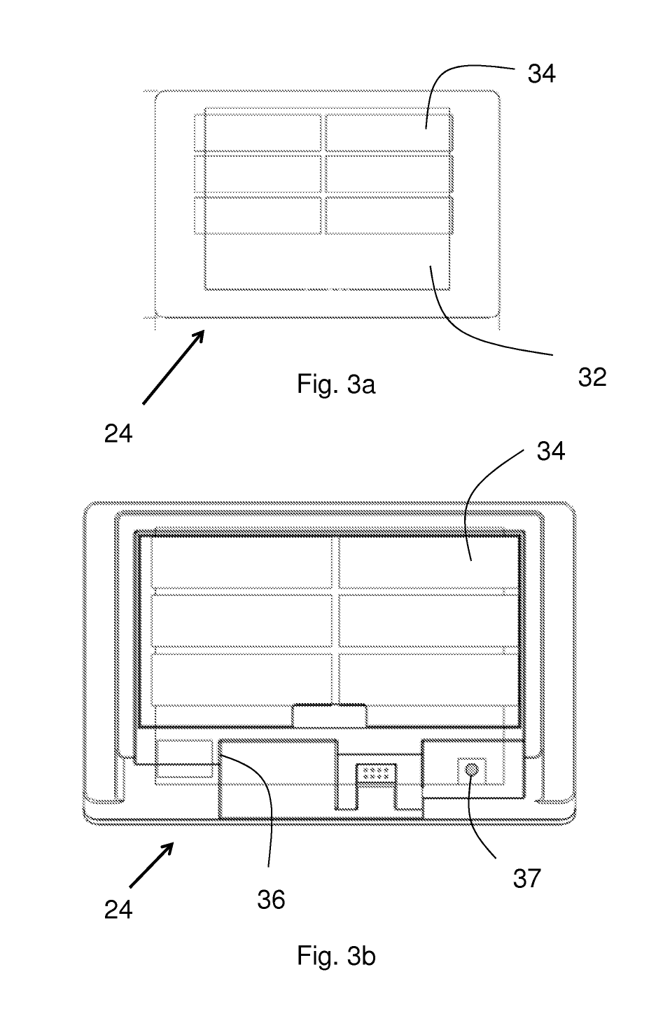

Referring to FIGS. 3a and 3b, in an exemplary embodiment of the present invention, the compliance device 24 comprises a patch antenna 32 towards the front surface of the device. In a specific embodiment, the patch antenna 32 has a 3-dB beam width of 60 degrees. This beam width gives the desired isotropic boundary for the present application and also provides a separation between the boundaries for adjacent compliance devices 24.

In one embodiment, the compliance device 24 also comprises a power connector 36 for connecting to a DC power supply to power the patch antenna 32 and the electronic circuitry. The compliance device 24 also comprises a battery compartment 34 for battery power.

In an exemplary embodiment, the compliance device 24 further comprises a reset button 37. When the reset button is pressed, the compliance device 24 is switched to a setup mode, allowing a technician to program the various parameters of the patch antenna 32 and other circuitry of the compliance device 24, for example through wireless communication via the patch antenna 32.

In another embodiment, the compliance device 24 further comprises an ambient light sensor. The ambient light sensor senses the light intensity of the environment, and can allow for different settings of the compliance device 24 based on the light intensity sensed.

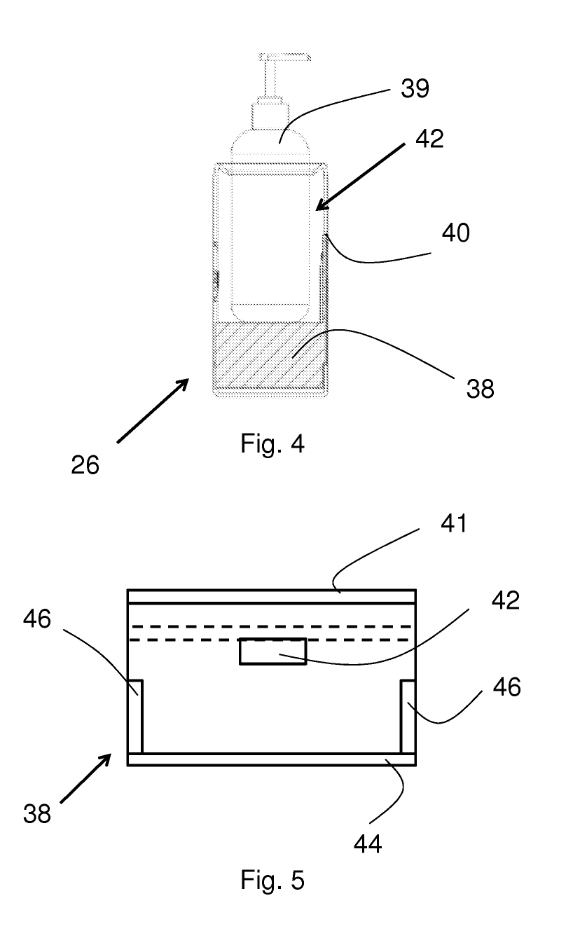

In an exemplary embodiment in FIG. 4, the action device 26 is a pump device for detecting a depression of a pump bottle 39. The action device 26 comprises a pressure-sensitive member 38 and an open-top housing 40 extending upwards from the sides of the pressure-sensitive member 38 and open at the top end. A pump bottle is adapted to be inserted through an opening 42 of the open-top housing 40, with the pump bottle resting on the top surface of the pressure-sensitive member 38.

In an exemplary embodiment as shown in FIG. 5, the pressure-sensitive member 38 comprises a movable platform 41 at the top surface thereof, a switch 42 underneath the movable platform 41, and a PCB board 44 with an omnidirectional antenna at the bottom end of the pressure-sensitive member 38. The movable platform 41 moves downwards from an initial position to a depressed position (as shown by the dashed lines) upon exertion of a downward pressure by the worker onto the pump bottle. The switch 42 is activated to send an action signal to the worker device 22 when the movable platform 41 is at the depressed position. The omnidirectional antenna ensures that action signal can be successfully transmitted to a worker device 22 at any direction relative to the action device 26. In an exemplary embodiment, the movable platform 41 is spring-biased such that it will revert to its default position after activation of the switch 42.

In an exemplary embodiment, the switch 42 is a dome switch that is physically depressed to be activated when the movable platform 41 is at the depressed position. In another embodiment, the switch 42 is a reed switch that is activated when the movable platform 41 moves or is at the depressed position. In this embodiment, a magnet is provided at the bottom surface of the movable platform 41 for activation of the switch 42, and the movable platform 41 in this embodiment does not need to physically touch the switch 42, so the switch 42 may be activated even the movable platform 41 is not entirely at the depressed position.

In an exemplary embodiment, the omnidirectional antenna on the PCB board 44 comprises a plurality of quarter-wave whip antennas 46 along the vertical direction. In a specific example, the action device 26 comprises four whip antennas 46 disposed on the four corners of the PCB board 44 (two of which are shown in FIG. 5). The PCB board 44 acts as a ground plane for the four whip antennas 46, such that the field is focused at an angle above the horizontal. In a preferred configuration where the worker device 22 is worn on the worker at a horizontal level higher than the whip antennas 46, having the field focused at an angle above the horizontal results in a better reception of the action signal by the worker device 22.

In a further embodiment, a clearance of at least 35 mm is provided between the base of the whip antennas 46 and the movable platform 40 at the depressed position. The clearance is to prevent the pump bottle from creating interference with the whip antennas 46 and optimize the signal strength at the desired angle.

In an exemplary embodiment, the plurality of whip antennas 46 is configured in a way such that only one of the whip antennas 46 is active at a given time. In a further embodiment, the plurality of whip antennas 46 activates and deactivates in a cycle with a single activation of the switch 42. The main advantage for such configuration is that interference caused by other antennas 46 can be safely ignored while still achieving horizontal omnidirectionality within a specific time frame. Installing the antennas 46 along the peripheral of the pressure-sensitive member 38 can also minimize the interference caused by other parts of the pressure-sensitive member 38.

In a specific example, a first whip antenna is activated to send the action signal twice upon activation of the switch. Afterwards, the first whip antenna is deactivated and the second whip antenna is activated to also send the action signal twice. This process repeats for each whip antenna, such that no matter what angle the worker device 22 is relative to the action device 26, there must be a whip antenna that at least sends a strong enough signal for the worker device 22 to receive.

In an exemplary embodiment, the pressure-sensitive member 38 is made as a waterproof block. That means the movable platform 41 is waterproof at any position and also during movement. By making the pressure-sensitive member 38 to be waterproof, the chance of any liquid, such as the content within the pump bottle, to affect the operation of the action device 26 e.g. shorting the electronic circuitry inside when the content is accidentally spilled onto the pressure-sensitive member 38 is minimized.

In one embodiment, the pressure-sensitive member 38 also comprises a battery compartment. In different embodiments, the battery can be replaceable or fixed, and the battery compartment can be located in any location internal or external to the action device 26.

In an exemplary embodiment, the base station 27 comprises an antenna, a processor, and data transmission components. The data transmission components can comprise a USB port, an Ethernet connector, or antennas for wireless transmission that can be the same or different as the antenna above, or a combination thereof.

In operation of the system, the worker device 22 is first distributed or provided to a worker. The worker attaches the worker device 22 to his chest through the attachment member 31. At the attached position, the patch antenna 28 of the worker device 22 is vertically oriented and facing away from the worker, therefore a maximum gain is achieved at the direction in front of the user.

The compliance device 24 is pre-installed at a predetermined location, for example at the head end of a hospital bed in a ward. The patch antenna 32 of the compliance device 24 is also vertically oriented at the installed position, facing towards the foot end of the bed. The compliance device 24 sends out monitoring signals at regular intervals, regardless of whether a worker device 22 is nearby. Similarly, the action device 26 is also pre-installed at a predetermined location with a pump bottle placed therein. Unlike the compliance device 24, the action device 26 only sends out action signals when activated.

When the worker attached with the worker device 22 enters a zone in proximity to the compliance device 24, hereinafter called the work zone, the signal strength of the monitoring signal received by the worker device 22 exceeds a first threshold. When the signal strength exceeds the first threshold, the worker device 22 will deem the worker to have entered the work zone. The worker device 22 then records the time of entrance into the work zone and the ID of the work zone in its memory, based on the information in the monitoring signal sent by the compliance device 24. In an exemplary embodiment, the information in the monitoring signal comprises a work zone ID or compliance device ID.

In an exemplary embodiment, the size of the work zone is a directional zone covering the hospital bed and around the hospital bed, but does not extend to an adjacent hospital bed. This is also called bed-level accuracy, meaning that there is at least one work zone dedicated to each bed, so the worker can be identified to be in proximity to a specific bed. The determination of the size of the work zone is based on the antennas of the worker device 22 and the compliance device 24, and also the signal strength of the first threshold.

As mentioned above, the compliance device 24 sends out monitoring signals at regular intervals. While the worker is within the work zone, every time the worker device 22 receives the monitoring signal, a timer related to the work zone will be refreshed. When the worker leaves the work zone, the signal strength of the monitoring signal received by the worker device 22 drops below a second threshold. The timer will no longer refresh when the signal drops below the second threshold, and the worker device 22 will deem the worker to have left the work zone when the timer expires. The time where the timer expires is then recorded in the memory of the worker device 22. In one embodiment, the timer related to the zone is also included in the monitoring signal sent from the compliance device 24 to the worker device 22.

In an embodiment, the first threshold is higher than the second threshold. A reason for this configuration is that the worker may move around the hospital bed or turn his body when taking care of a patient, and such movement may reduce the signal strength received slightly. However, such movement should not be determined as the worker exiting the work zone.

Alternatively, when there are multiple compliance devices 24 in the system, if the signal strength received from the compliance device 24 of the work zone the worker is currently in falls below the signal strength received from another compliance device 24 related to another work zone, the worker is also deemed to have left the current work zone.

When the worker depresses the pump bottle, the action device 26 sends the action signal to the worker device 22. The action signal includes an ID of the action device 26 and the worker device 22 records the same in the memory therein with a timestamp of receipt of the signal. In one embodiment, the worker device 20 determines the strongest signal transmitted among the antennas 46 of the action device 26 when determining whether the worker is proximate the action device 26 during the time of depression.

After the worker device 22 obtains the above information, a compliance rate, in this case a compliance rate of the worker performing hand hygiene before touching the patient, is determined from the time information through a predetermined rule, for example a look-up table or other known methods. In an embodiment, the predetermined rule is made based on a guideline issued by the World Health Organization. In general, if the action device 26 is activated between exiting a work zone and entering another work zone, it is likely that the worker has washed his/her hands, i.e. complied with the protocol.

In an exemplary embodiment, a worker will be alerted when the system determines that he/she does not wash his/her hands, or does not satisfy a compliance rate requirement in a predetermined period of time, therefore the worker will be reminded to be more careful in the future, thus reducing medical error. In one embodiment, the worker device 22 comprises an indicator such as an LED, a buzzer or a vibration motor to alert the worker when the system determines that he/she is out of compliance.

In an exemplary embodiment, the information above is sent from the work device 22 to a backend server through the base station 27, and the compliance rate is determined at the backend server. In another exemplary embodiment, the worker device 22 is equipped with a processor to determine the compliance rate therein, and the compliance rate is sent to the base station 27 for record. This embodiment enables real-time alerting of the worker as the worker does not need to move in range of the base station 27 for determination of the compliance rate.

In an exemplary embodiment, the data stored in the worker device 22 is cleared after forwarding to the server through the base station 27, to ensure no repetitive data will be sent to the base station 27 and also allowing more updated information to be stored in the worker device 22.

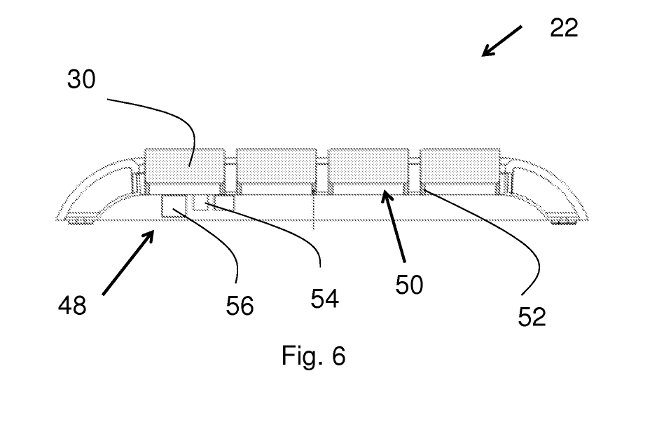

In an exemplary embodiment, the system further comprises a battery charger 48. Referring to FIG. 6, the battery charger 48 comprises a plurality of slots 50 on its upper surface for insertion of the battery 30 of the worker device 22. In a further embodiment, a plurality of stoppers 52 is provided within the slots 50 to ensure proper alignment of the battery 30 to the battery charger 48 for recharging the battery 30. The battery charger 48 also comprises a power connector 54 for connection to a power outlet through an adaptor.

In an exemplary embodiment, the functionality of the base station 27 is integrated into the battery charger 48, i.e. the base station 27 and the battery charger 48 are the same device. As such, the battery charger 48 also comprises data communication ports such as Ethernet cable port 56.

In an exemplary embodiment, a plurality of contacts (not shown) is provided within each slot for contacting the battery 30 at the aligned position. In a further embodiment, the battery 30 contacts the battery charger 48 and the worker device 20 at the same locations, ensuring that the battery 30 must be detached from the worker device 20 during recharging. In another embodiment, the battery 30 is charged through induction thus contacts are not necessary.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, the action device 26 can be used to detect activation of other devices other than a pump bottle, such as a tap, a paper towel dispenser or hand dryer etc. Depending on the way of activation, the pressure-sensitive mechanism can be changed to sense movement, heat, or any combination of the above.

What is claimed is:

1. A system for reducing medical error comprising:
a) a worker device adapted to be worn on a worker, having:
i) a directional antenna working as a transmitter and a receiver;
ii) a battery; and
iii) a memory for storing data;
b) a compliance device having a directional antenna working as a transmitter, said directional antenna defines a work zone relative to said compliance device based on a signal strength received by said worker device from a monitoring signal transmitted from said compliance device;
c) an action device adapted to be installed to a pump bottle, having:
i) a pressure-sensitive mechanism for actuating said action device upon said worker pressing said pump bottle;
ii) an omnidirectional antenna adapted to transmit an action signal to be received by said worker device upon actuation of said action device; and
d) a base station adapted to receive said data transmitted from said worker device.

2. The system according to claim 1, wherein said battery of said worker device is rechargeable through a battery charger.

3. The system according to claim 2, wherein said battery charger comprises a slot for insertion of said battery of said worker device, wherein at least one stopper is provided within said slot to align said battery to said battery charger.

4. The system according to claim 1, wherein said battery of said worker device is detachable from said worker device.

5. The system according to claim 1, wherein said antenna of said worker device is a patch antenna oriented vertically to achieve horizontal directionality.

6. The system according to claim 1, wherein said antenna of said compliance device is a patch antenna oriented vertically to achieve horizontal directionality.

7. The system according to claim 1, wherein said antenna of said action device comprises a plurality of quarter-wave whip antennas oriented vertically in a circularly symmetric configuration to achieve horizontal omnidirectionality.

8. The system according to claim 7, wherein said plurality of whip antennas are configured in a way such that only one of said plurality of whip antennas is active at a given time.

9. The system according to claim 1, wherein a ground plane is located at a bottom end of said antenna of said action device.

10. The system according to claim 1, wherein a distance of at least 35 mm is provided between a ground plane of said action device and a top surface of said pressure-sensitive mechanism.

11. The system according to claim 1, wherein said pressure-sensitive mechanism comprises a movable platform adapted to move to a depressed position upon exertion of pressure, said movable platform at said depressed position activates a switch for transmitting said action signal.

12. In a system according to claim 1, a method for reducing medical error comprising:
a) providing said worker device to a worker;
b) detecting said worker entering said work zone based on said signal strength of said monitoring signal;
c) detecting activation of said pump bottle by said worker based on said action signal;
d) detecting said worker exiting said work zone based on said signal strength of said monitoring signal; and
e) determining a compliance rate based on details of steps b), c) and d).

13. The method according to claim 12, wherein said step c) comprises the steps of:
a) sensing a pressure exerted on said pump bottle by moving a movable platform of said pressure-sensitive mechanism to a depressed position; and
b) activating a switch for transmitting said action signal to said worker device when said movable platform is at said depressed position.

14. The method according to claim 12, wherein said pump bottle comprises a plurality of whip antennas, said step c) further comprises the step of configuring said plurality of antennas such that only one of said plurality of antennas transmits said action signal at a given time.

15. The method according to claim 12, wherein said step b) comprises the step of detecting said worker entering said work zone when said signal strength of said monitoring signal exceeds a first threshold, and said step d) comprises the step of detecting said worker exiting said work zone when said signal strength of said monitoring signal drops below a second threshold, wherein said first threshold is higher than said second threshold.
